(19) United States
(12) Patent Application Publication
(10) Pub. No.: US 2022/0346652 A1
(43) Pub. Date: Nov. 3, 2022

(54) WEARABLE FOOTWEAR SENSOR ARRAYS FOR DETECTION OF CARDIAC EVENTS, BODY MOTION, AND MUSCULAR ACTIONS

(71) Applicants: The Regents of the University of California, Oakland, CA (US); Tacsense, Inc., Woodland, CA (US)

(72) Inventors: Tingrui Pan, Woodland, CA (US); Benjamin Arthur Bazor, Davis, CA (US); Zhichao Zhang, Woodland, CA (US)

(73) Assignees: The Regents of the University of California, Oakland, CA (US); Tacsense, Inc., Woodland, CA (US)

(21) Appl. No.: 17/282,188

(22) PCT Filed: Apr. 16, 2019
(86) PCT No.: PCT/US2019/027754

§ 371 Date: Apr. 1, 2021

Related U.S. Application Data
(60) Provisional application No. 62/742,209, filed on Oct. 5, 2018.

(51) Int. Cl. A61B 5/0205; A61B 5/021; A61B 5/024

(52) U.S. Cl. CPC A61B 5/0205; A61B 5/02116; A61B 5/02438; A61B 5/02444; A61B 2562/0219

(57) ABSTRACT

A foot-based wearable system disposed proximate to the dorsalis pedis artery can detect cardiac and muscular activities. Utilizing flexible iontronic sensing (FITS) technology, a sensing array detects both cardiovascular functions, such as heart rate, ECG, and respiration and motion artifact signals with a spatial reference to muscular activities based on the orientation of the array. Individual tendon responses are analyzed and correlated to different pedal gestures, from which multi-channel signals can be used to distinguish different activities. Wearable articles of the invention include a platform to simultaneously analyze both vital signals and body activities from the cardiac waveforms and muscular responses in a natural and unnoticeable fashion. The data-collecting wearable system provides a means to assess personalized health and daily activities on a continuous basis.

### BACKGROUND

This application claims priority to U.S. Provisional Application 62/742,209, filed Oct. 5, 2018, entitled “Flexible Iontronic Sensing Wearable Detects Pedal Pulses and Muscular Actives,” which is specifically incorporated by reference herein.

Wearable devices provide an intimate physical and data interface between the human body and mobile computing devices. As medical technology has developed, several body signals have been monitored for data such as gestures, footsteps, general activity levels, and other events have been measured by body sensors and converted into electronic signals that are translated into physiologic data and displayed to a user. Additionally, cardiovascular events such as pulse, blood volume variations, blood oxygen level, electrocardiograph (ECG), electroencephalograph (EEG), and respiration have been successfully measured and analyzed through the wearable sensor interfaces.

One challenge of the wearable device is to identify an ideal body location to receive a relatively long-term or repeated attachment. Researchers have tested a number of sites on the human body (e.g. head, chest, upper arm, wrist, waist, fingers, and feet) to attempt to locate an ideal site that is both useful for monitoring data and for which sensors can be readily attached without interfering with normal day-to-day activities. For example, the chest has been used to acquire ECG, heart rate and respiration signals with a chest band-based heart monitor. Finger rings have been used as a simple healthcare monitor to detect pulse, blood oxygenation, and blood volume variations. Wrist-based sensors incorporated into a wristwatch type device have likely been the most investigated site due to the traditional historic practice of wearing wrist watches in the ability to acquire cardiac signals including pulse and blood volume variations from arteries that are readily detected in the wrist.

Despite these advances, a number of limitations are still present. Poor long-term wearability is the major challenge that prevents many wearable technologies from being widely adopted because the sensor arrays that are available to measure large amounts of data, and to measure them accurately, are bulky and simply uncomfortable to wear. The sensors and the materials holding the sensors in place can be rigid, restrictive, irritating and unable to secure a long-term attachment. As a result, an ideal match between body location and long-term comfort is still being sought.

Human feet contain abundant vasculature, strong tendons and muscles, and characteristic bone structures , from which valuable health information and muscular activities can be continuously collected and precisely extracted. However, although human feet have been considered as an ideal location to place wearable sensor devices, the existing footwear technologies focus on activity tracking and measurement of mechanical contact forces, such as steps and weight bearing, together with some pulse and artery monitoring in clinical environments, and often requiring measurement at other locations on the body. Specifically, microchip-enabled accelerometers have been used in shoes or mounted as shoe clips to record steps during exercises, or detect kinematic changes of gait cycles. These devices and method have been used in the ports footwear industry to develop smart athletic shoes (e.g., Nike+®, Adidas Micropacer®, and Under Armour First Run®.

In terms of the mechanical contact force measurements, the ground reaction force (GRF) can be detected by the flexible pressure sensors embedded in the insole and has been used to map the plantar pressure distribution, as well as to conduct gait analysis. Recently, researchers group illustrated a method to apply ballistocardiogram (BCG) for heart rate detection by incorporating piezoelectric films into the insole, although the BCG signals were rather weak to be detected until the participants did a special stepping training to enhance their cardiac outputs See Chen M. Liu, F. Jiang, H. Jiang, S. Ye, and H. Chen, “Low-power, noninvasive measurement system for wearable ballistocardiography in sitting and standing positions,” Comput. Ind., vol. 91, pp. 24-32, Oct. 2017.

Therefore, although attempts have been made to use sensors around the human foot to measure important physiological parameters, limitations on the sensor arrays and the ability to incorporate sensors into wearable footwear have prevented significant advances in sensor arrays incorporated into footwear and used to measure a wide range of physiological parameters.

### SUMMARY OF THE INVENTION

This invention includes a foot-based wearable system that can detect cardiac signals, cardiac data, physiologic data, motion artifacts, artifacts of muscle and tendon motion, and signals that reflect muscular activities that can be translated into specific gestures. To achieve such functions in a wearable and comfortable item of footwear, a plurality of flexible iontronic sensing (FITS) pressure sensors are specially configured and integrated into a pre-determined array configuration to create a pressure sensor array comprised of a plurality of sensors that can be integrated into traditional footwear or footwear that has been specially configured to receive the sensor array and optionally accompanying circuit components.

Although the human foot presents a number of options for measuring pressure and physiologic data, the present invention includes selection of the configuration of the array together with the specific performance parameters of the individual sensors and the overall performance of the sensor array to be specifically targeted to the region of the foot adjacent to the dorsalis pedis artery. Accordingly, the sensor array of the present invention takes advantage of both the orientation of the array around the dorsal artery of the foot together with high performance features of the individual sensors, including high flexibility, comfort, close conformity to the skin, or to a clothing or fabric layer in close conformity to the skin, high pressure-to-capacitance sensitivity, high signal-to-noise ratio, the ability to compensate for background and noise artifacts, fast response time, and sensing range typical to shoe and cardiac pressure. The sensor array is integrated into a wearable article of footwear and the sensor array assembly is integrated into the footwear such that the sensor array is adjacent to the artery such that multiple physiological parameters are detected.

The sensor array has a plurality of individual sensors that, by integration into the footwear assembly, has the capability to measure multiple and independent cardiac functions, physiological functions, muscle and tendon artifacts, and coordinated foot gestures based on characteristic signals received from the pressure signals at individual sensors of the array. The individual sensors can be separately or collectively selected for signal processing that includes comparing individual sensors to a cardiac data profile that identifies an individual sensor in the array that is receiving a stronger signal correlated to cardiac function compared to other sensors in the array. The profile of the cardiac data contains information to match the sensed data, such as time interval, amplitude of the signals and frequency of the signal and a normal heart rate range from a large population. In this configuration, this particular sensor is identified as a primary signal source for cardiac function and is so designated for a single sensing session.

Once a primary cardiac signal is identified, the known spatial configuration of the individual sensors in the array can be used to identify a secondary sensor, separate from the primary cardiac sensor, to sense any of additional cardiac data, separate physiological signals, including muscle and tendon artifacts and essentially any other differential pressure measurement detectable across the individual or plurality members of the array.

Because of the flexible nature of the sensor assembly and the ability to tailor the configuration of the individual sensors in the array to the area adjacent to the dorsalis pedis artery, the sensor array can readily be integrated into an article of footwear. Footwear includes any variety of sock, shoe, or other garment that conforms closely to the foot such that the array can be maintained in a stable position near the dorsalis pedis artery.

Ideal locations for the sensor array include the inner top surface of a shoe or socket, including specifically the tongue of tissue or any structural elements of issue that takes the position equivalent to the tongue in an ordinary athletic shoe. When worn, the sensor array is integrated into the article of footwear and then is maintained in a stable position during the time that the footwear is worn by the user. This garment must produce some pressure to place the sensor into conformal contact with the foot. In some embodiments the pressure is fixed due to the elasticity of a sock, or fly-knitted shoe cover, is manually adjustable through Velcro, straps, and laces, or is automatically adjustable through motorized laces or self-pumped airbag. Notifications can be given to the user to adjust the tension for optimal detection of physiological signals. Because of individual variations, the positioning of the array might vary from time to time depending on individual variations by the user. The array is capable of sensing these variations and the individual sensors can be re-configured electronically to accommodate such variations while collecting data that can be compared with prior uses of the device and integrated into a data set that compares the data secured from the sensor over a large number of individual sessions by the user.

FITS devices offer advantages in pressure sensing, with high sensitivity, excellent mechanical ruggedness, and reliable flexibility, due to the ultrahigh interfacial capacitance and fast polarization of the iontronic materials. The array disclosed herein is made from a solid-state flexible ionic coating in an elastic contact with a conductive electrode array, which can measure pressures in the device at a sensitivity of up to 1 nF/mmHg with a detection range of 1 to 200 mmHg. Resolution is in the range of 0.01 mmHg to 1 mmHg (with 0.01-0.05 mmHg most preferred), and sensitivity is in the ranges of 0.01 nF/mmHg to 1 nF/mmHg (with 0.02-0.1 nF/mmHg most preferred). Device response time is in the range of 0.1 to 10 milliseconds which provides the ability to detect micro fluctuations in blood pressure, rapid changes in cardiac signals, and to precisely determine the time of cardiac events (e.g. systolic peak, dicrotic notch, inter-beat interval). With such a high sensitivity, small variations of blood pressure can be detected at one or more sensors in the array and compared with a stored cardiac profile like data segments, empirical values or reference values to identify an individual sensor producing a primary cardiac signal from the dorsalis pedis artery, which is also known as the pedal pulse waveform, in a gentle contact with the foot or foot covered with a certain fabric layer around the baseline pressure exerted by the shoe or sock of approximately 10 mmHg. Alternatively, the primary cardiac channel can be determined using signature properties from the standard cardiac profile such as the peak-to-peak intensity, onset time, and regular period, which can be characterized through various mathematics and physiological knowledge commonly understood within the art.

Because of the ultrathin and flexible construct of the sensing array, the range of 50 μm to 2 mm in thickness. The array can be placed in a position adjacent to the dorsalis pedis artery and in incorporated into an article of footwear in contact with the dorsal area of the foot. Because the array is comprised of a plurality of individual sensors, the orientation of the individual sensors in the array preferably has a known spatial configuration such that individual distances and relative positions from sensor-to-sensor are known as described above. Given the known physiology of the foot, and even taking into account individual person-to-person variations, the signal data obtained from an individual sensor or a plurality of sensors can be used to correlate the spatial relationship of one or more sensors to an additional singular sensor, or set of sensors, so that specific cardiac signal data, or physiological artifact data, can be correlated to additional cardiac or physiological function data obtained from separate sensors and having a distance and direction component based on the known spatial configuration of the array. Calibration procedures conducted by the user can compensate for person-to-person differences and improve the accuracy of physiological feature mapping and parameter determination. A calibration procedure can, for example, consist of conducting various foot gestures in a specified sequence, recording signals for the pressure sensor array, correlating those signals to the specific foot gestures, retaining a collection of signals in device memory to compare with future signals, and correlating the calibrated values against signals from a measured user gesture to identify the gesture.

Because the dorsalis pedis artery region of the dorsal area of the foot has a known configuration, the sensor array advantageously places a number of individual sensors, and a predetermined number of sensors within a defined area. Advantageously, there are at least 2 sensors within 3-10 centimeter squared area, and preferably with a 3, 4, or 5 square centimeter area. Furthermore, at least one or more of the individual sensors is always placed at least 200 pm every adjacent sensor. In a preferred embodiment, a first primary sensor designated as the primary cardiac sensor is a distance at least 200 μm, or at least 300 μm 400 μm or 500 μm no more than 3 mm, 4 mm, or 5 mm, and preferably between 200 um to 2 mm from an adjacent sensor. The adjacent sensor obtains a pressure signal generating a measure of a physiological function detected in the human foot and selected from static pressure distribution, and the group of motion or position artifacts correlated to any of tendons, muscles, bones, cartilage or ligaments, or combinations of any or all of the above. Also, a plurality of sensors or individual sensing arrays can be arranged in a honeycomb (or similar) pattern where rows of sensors are offset such gaps between sensors are covered in the next row of sensors, allowing all locations in the transverse direction of the foot to be covered. As described herein, the sensor obtaining a signal from a cardiac function is sometimes described as the “first” or “primary” sensor while the additional sensors obtaining an additional cardiac signal or physiological signals may be deemed the secondary or second sensor the designation is arbitrary in terms of the order in which the signals are received or analyzed and the placement of the individual sensor in the array. The only operational requirement is that at least one single sensor is analyzed and identified as providing a signal from a cardiac function and so designated in the overall operation of the array.

The invention also includes specific modes and methods of analysis based on the individual signals obtained from one or more pressure sensors in the sensor array. A unique feature of the invention includes the individual selection of pressure sensors in the array in a defined order to individually isolate and compare cardiac function or other physiological parameters signals obtained from the array. In a preferred embodiment, the method includes selection of a first sensor producing a primary cardiac signal, followed by selection of a second, separate sensor yielding a secondary cardiac, or other physiological parameter. Each sensor will detect pressures from different sources including: static/baseline pressure between foot garment and foot, inertial forces from movement causing separation or joining of foot garment and foot, cardiac pressure from the blood pulsing through an artery, or pressures exerted through the movement of tendons, muscles, or bones whether due to contraction or rearrangement during foot gesture. Each pressure signal has a unique signature in intensity, space, and time such that they can be isolated to specific sensors (e.g. primary cardiac signal) or events (e.g. foot gesture). Linear algebraic combinations and other signal processing techniques such as Principle Component Analysis (PCA), adaptive noise cancellation, or machine learning algorithms can be used to isolate pressure signals from each other.

For example, the static pressure on a reference sensor signal can be subtracted from the primary cardiac signal to determine the pure cardiac pressure signal. Similarly, common inertial forces can be subtracted to denoise the cardiac pressure signal during movement. Simple analysis of the primary cardiac signal (or other physiological feature) places the location directly at the center of the sensing element limited in spatial resolution to the distance between sensing elements (pitch). Mathematical techniques can allow for locating cardiac signal at a higher precision than the sensing pitch using a combination of the location and intensity of the primary, second, and any other detectable cardiac signals.

In one embodiment of the invention where the system is worn on each foot, the common origin of the cardiac signal produces synchronous responses in both systems. This effect can used to produce motion-resistant pure cardiac signals by detecting signal on the system with the minimum noise or combining signals using aforementioned signal analysis techniques. For example, during standard walking, stance and swing phases alternate. By selecting the system in the swing phase (with one foot airborne) as the primary cardiac signal, the motion artifacts are significantly reduced.

Specifically, after one sensor is selected from the array as yielding a primary cardiac signal based on pulse waveform-and is preferably analyzed against a stored or accessed cardiac profile, critical additional signals measuring additional or repeated cardiovascular parameters are collected from an additional, separate sensor. The designation of a sensor as first, second or separate does not necessarily require that these individual sensors were the first or second sensors in order analyzed for signal or measurement, but rather that these were designated as first or second based on substantive analysis of the signal detected and so designated for further signal processing. Accordingly, in, for example, a ten-sensor array, the 9^{th} signal analyzed in sequence may be the signal that is matched to a cardiac signal and designated as a “first” signal for subsequent analysis and comparison with other signals. The “first” signal is sufficient to determine several cardiac functional parameters, such as upstroke time, augmentation index, blood pressure trend, as well as derived vital signs, such as heart rate (HR), heart rate variability (HRV), and respiratory estimation.

The additional, separate or second sensor is similarly selected based on the measured signal of an additional physiologic parameter, including additional cardiac functional parameters, such as upstroke time and augmentation index, as well as derived vital signs, such as heart rate (HR), heart rate variability (HRV), pulse wave velocity, and pulse transit time, and respiratory estimation. The second, separate signal obtained from any literal sequence or order of analysis, is designated and identified as yielding a secondary or separate signal that can be further processed and compared with both the primary cardiac signal as well as additional tertiary or quaternary signals from the array. As noted above, because the spatial relationship between each of the sensors of the array is predetermined and known relative to one another in advance of the signal detection sequences, all of the measurements from the first, second, or additional pressure sensors can be correlated to specific physiological structures that are unique to each of the individual sensors, the remaining sensors in the array, and the orientation relative and proximal to the dorsalis pedis artery. Separate signals correlated to specific physiological structures, such as tendons, can allow for individual tendon contract/relaxation analysis to determine foot gesture, gait patterns, or other biomechanical parameters of foot status and motion.

The preferred embodied of the invention uses FITS devices for detection of high quality primary cardiac signals. The signal quality allows for detection of aforementioned cardiac functional parameters such as HRV and can provide tolerance for person-to-person variability. The high accuracy measurement of HRV can be used to derive several physiological-related parameters for stress evaluation. For example, frequency analysis parameters such as the integrity of the high frequency band (HF), low frequency band (LF) ultra low frequency band (ULF), and ratio of LF to HF as described in Kim,Hye-Geum et al., “Stress and heart rate variability: a meta-analysis and review of the literature.” Psychiatry Investigation 15.3 (2018): 235. Other flexible pressure sensors may be used detect the primary cardiac signal at lesser quality and used to calculate noise-tolerant derived vital signs such as heart rate, whereas low magnitude signals such those used for augmentation index are challenging without proper signal-to-noise ratio and resolution. Examples of other flexible pressure sensors include: piezoresistive, piezoelectric, capacitive, and pneumatic manometry systems. Combination with non-pressure sensors such as accelerometers, gyroscopes, magnetometers, temperature sensors, and humidity sensors can provide additional data useful in noise reduction or physiological parameters.

In one particular structure and method of the invention, the array of the invention yields a signal pressure set of signals, at least one of which is been compared with a standard 2-lead electrocardiogram (ECG) that has been stored as a reference or is generated in real time from the sensor array of the invention. Furthermore, the linear array with multiple sensing units is adjacent to the dorsalis pedis artery thereby covering the transverse plane on the dorsum, making the capture of pedal pulse signals without any special alignment step, while the location of pedal artery provides a spatial anatomic reference to muscular activities. Finally, muscular responses are collected with a sufficient resolution to track individual tendon activities, providing a highly integrated way for foot gesture classification, see J. Alexander, T. Han, W. Judd, P. Irani, and S. Subramanian, “Putting Your Best Foot Forward: Investigating Real-world Mappings for Foot-based Gestures,” in Proceedings of the SIGCHI Conference on Human Factors in Computing Systems, New York, N.Y., USA, 2012, pp. 1229-1238, gait analysis M. W. Whittle, “Clinical gait analysis: A review,” Hum. Mov. Sci., vol. 15, no. 3, pp. 369-387, June 1996, as well as body status tracking.

A highly sensitive and flexible pressure sensing array is enabled by the novel iontronic sensing principle, sensor array and method of sensing as described herein. The array has been fabricated and oriented in combination with support structures to simultaneously acquire body vital signals and track pedal skeletomuscular activities. The unique characteristics of the array allow it to be seamlessly integrated in a footwear format, such as a shoe of any type, a sock or any article of clothing that maintains the sensor array adjacent to the dorsalis pedis artery. The device illustrates that a wearable device can capture high-resolution peripheral arterial pulse waveforms, from which both heart rates and respiratory patterns can be extracted within a medical-standard precision.

The pressure sensor array is electrically connected to a data module that may be placed separately on an article of clothing or incorporated into a support structure that is also wearable on the foot. The data or circuit module contains or is connected to a processor that contains instructions for processing signal data, including shoes and associated articles.

Moreover, the high-spatial resolution of the sensing array allows alignment-free capture of pulse signals as well as provides a spatial reference to the pedal structures. It further enables tracking of individual pedal tendon movements, from which the majority of foot gestures can be assessed in real-time. The device operates as a personal mobile platform to acquire and analyze the human health and activity information in a comfortable and unnoticeable fashion and that is integrated into ordinary articles of clothing without great expense or interference with ordinary functions.

### DESCRIPTION OF THE DRAWINGS

FIGS. 1A-1D are the sensor array of the present invention disposed adjacent to the dorsalis pedis artery and to surrounding physiological structures suitable for measurement by the array.

FIG. 1A illustrates a sensor array sensing region that is located laterally above the navicular bone and three cuneiform bones. The physiology of the foot proximate to the dorsalis pedis artery and in FIG. 1B a cross section about plane A is shown including the four major tendons (TA, EHL, EDLS, and FT) and underlying bone, connective tissue, subdermal layers and vascular structures that are adjacent to the dorsalis pedis artery and are susceptible of generating a static or dynamic pressure signal measured by the sensors of the array disposed generally in the dorsum region adjacent to the dorsalis pedis artery.

FIG. 1C shows the disposition of the sensor array relative to an outline of an article of footwear. The labeled regions are traditional regions of the associated footwear. FIG. 1D is an isolated view of the sensor array shown in FIG. 1A including one option for the orientation of the individual sensors in the array and a dedicated electrical connection for each sensor in the array sensing region. The electrical leads for each sensor are oriented in a low profile planar array that does not interfere with the use of the article of footwear.

FIG. 2A is the device architecture of a flexible iontronic sensing unit (FITS) before and during application of a mechanical load. FIG. 2B is an equivalent circuit model of the FITS device. FIGS. 2C-2E are characterization of the electromechanical sensitivity of the FITS under various geometric parameters: FIG. 2C—Sensitivity curves: radius of the sensing chamber (5 mm, 3.75 mm, 2.5 mm, and 1.25 mm); FIG. 2D—Sensing membrane thickness (25 μm, 50 μm, and 75 μm); and FIG. 2E—Spacer layers height (20 μm, 50 μm, and 100 μm).

FIG. 3 is an article of footwear having the sensor array oriented adjacent to the dorsalis pedis artery, together with one example of the orientation of a circuit module case and the other structural elements of an item of traditional footwear. In this embodiment, the article of footwear has been altered to accommodate the sensor array and circuit module of the invention.

FIG. 4A is a 5 unit sensor array component and a circuit board is comprised of an analog front with five low-voltage operational amplifiers, an 8-bit MCU with ADC component, a Bluetooth low energy module and a power management module with a standard rechargeable Lithium-ion battery. FIG. 4B is a block diagram of the circuitry and the system integration of the sensor array in a regular athletic shoe, where the 6 blocks are listed by function and components of signal detection, signal processing, power management, and data processing for ultimate display to the user.

FIG. 5 is an analytical flowchart showing the progression of sensing steps leading to separate detection of pressure signals for cardiac/pulse and motion analysis.

FIGS. 6A-6C are a series of graphical use interfaces showing several cardiac and physiological parameters measured by the sensor array and displayed to the user.

FIGS. 7 is a typical high-resolution pulse waveform recorded from the pulse-sensing unit in the device, from which the respiratory patterns can be extracted from the waveform envelope.

FIG. 8 is a detail of an isolated high-resolution pulse waveform from FIG. 7 indicating at points i-v: i) diastolic uprising, ii) systolic peak point, iii) systolic decline, iv) valley and v) peak of peripheral dicrotic notch, respectively.

FIG. 9 plots the simultaneously recorded the pulse waveform (bold) and the ECG waveform (fine). The two signals have a correlation coefficient of 0.97.

FIG. 10 shows five constituent signals generated from the sensor array located adjacent to the of the dorsalis pedis archery, graphs of the variation in the progression of the signal over time. In this example, the unique constituent signals are analyzed and correlated to profiles indicating motion(s) of the foot such as dorsiflexion, plantarflexion, eversion, and inversion.

### DETAILED DESCRIPTION OF THE INVENTION

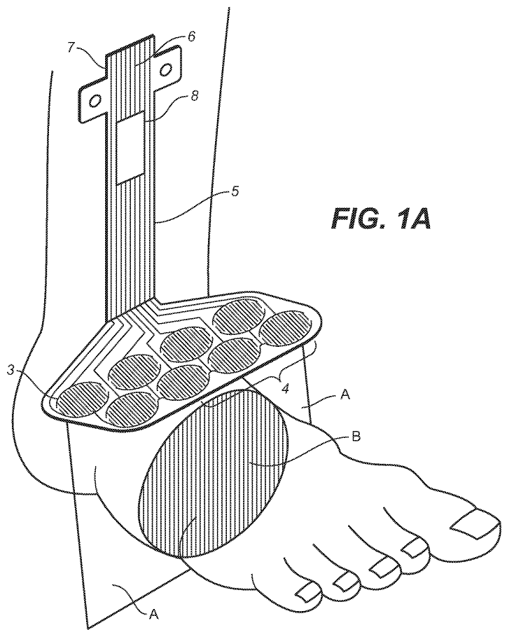

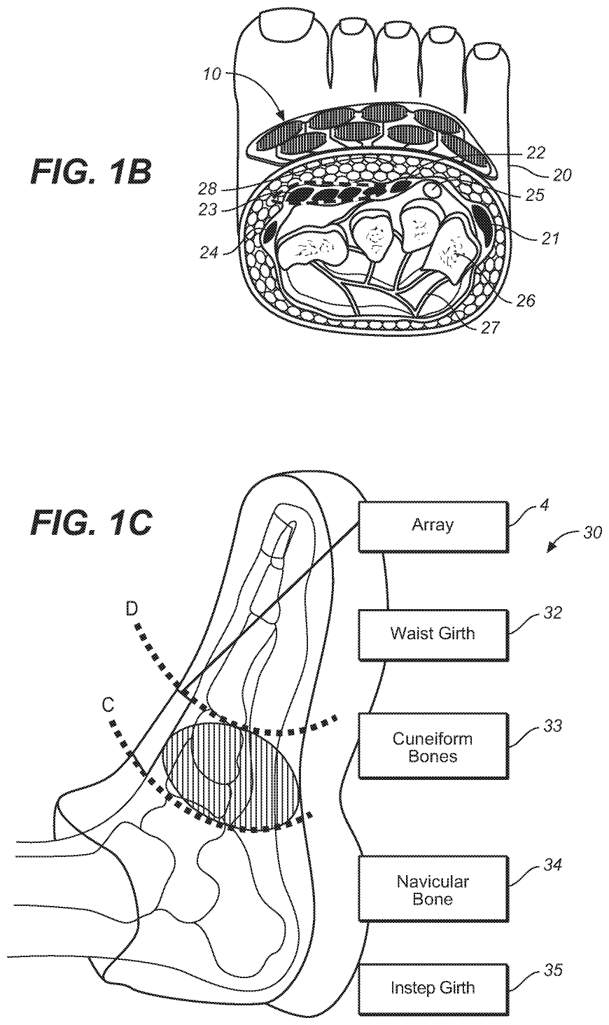

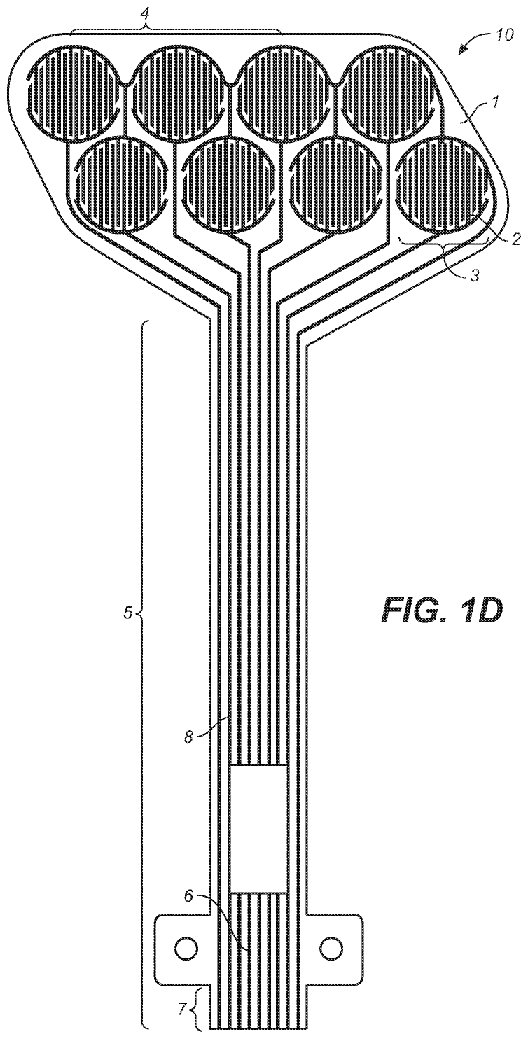

Human feet contain rich vasculature and characteristic bones and muscles that enable a wide range of motion and activities that sustain daily living. The foot also withstands incredible forces and retains strength and flexibility through a wide range of functions such as standing and walking. Because the foot is so intimately involved in a wide range of human activities, it is an ideal location to measure the cardiac and physiological functions that accompany these activities. Two major arterial branches passing towards the foot, i.e., dorsalis pedis artery and posterior tibial artery. The pulsations of both arteries can be directly detected and are known as an indicator of peripheral vascular health. The present invention takes advantage of the ability to locate a sensor array adjacent to the physiologic structures as shown in FIG. 1A. Referring specifically to FIG. 1A, the sensor array component 10 of the invention is comprised of a substrate 1 and iontronic sensing element 2, an individual and operational iontronic pressure sensor 3, and the assembled set of individual sensors displaced in the substrate 1 to form the sensor array 4. The structure of the array component 10 is detailed in FIG. 1D herein and accompanying text below.

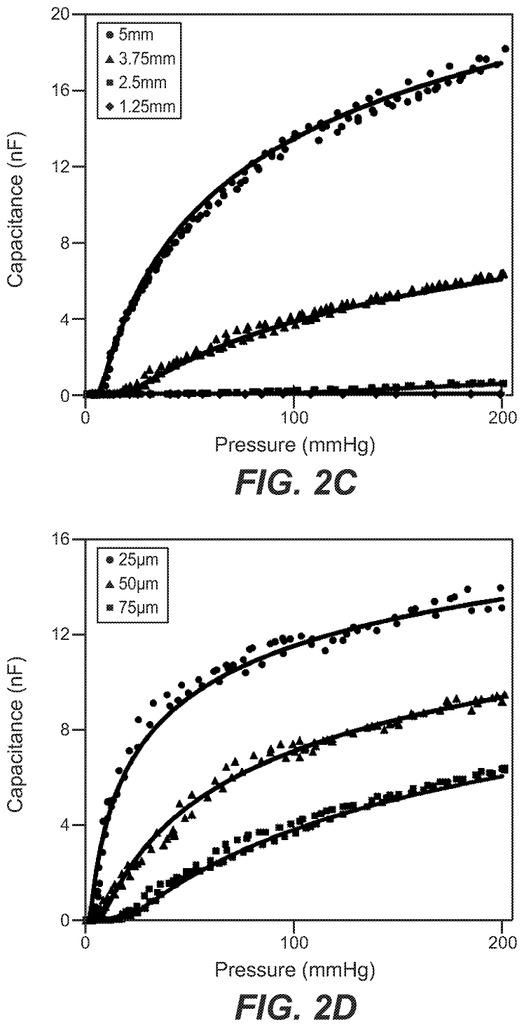

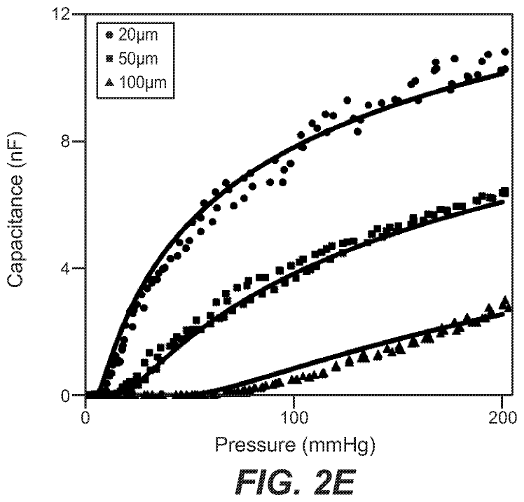

A portion of the array component 10 is comprised of a segment of substrate material 5 wherein individual electronic leads 6 are disposed to electrically connect the individual sensors 3 to a connector 7 that facilitates electrical connection to a separate a circuit module (not shown—see FIGS. 3 and 4 below) for processing pressure signals obtained from the sensor array 4. Accordingly, the array may be viewed as a configuration of individual sensors 3, collected and assembled to form the array 4 and having an electrical connection of a variety of different individual configurations that terminates with an electrical connection. An example of a separate sensor or array component 8 is comprised of only the sensor array, substrate and electrical connectors that can be used and sold separately to be plugged in to a suitable item of footwear or circuit module that has all of the other components of the overall system and offers the separate sensor array component as a replaceable article. Alternatively, the entire sensor array component 10 can be permanently or semi-permanently integrated into an article of footwear that is specially designed to receive the sensor array component 10. In such an embodiment, the connector 7 might be replaced with a continuous run of the individual electrical leads 6 connecting to the data processing circuit module.

Referring to FIG. 1B, portions of the human anatomy adjacent to the dorsalis pedis artery 25 are illustrated to demonstrate that region of the anatomy adjacent to the dorsalis pedis artery 25 are susceptible of being measured by the sensor array component 8,10 when positioned adjacent to the artery 25. Generally, the dorsum region of the foot is comprised of dorsalis pedis artery 25 and a group of four parallel tendons (as described below) that can be found between the skin and bones, underneath sub-epidermal skin layers 28 and with surrounding muscular 29, nerve and connective tissue (not shown). The complex arrangement of these structures enables the remarkably wide range and types of motion of which the human foot is capable. Because different motions of the foot involve different movement of muscle, tendon and, bone, the high sensitivity and accuracy of the sensor array component 10 enables the measurement and characterization of these motions through differences in pressure measured at the dorsum region of the foot when the sensor array component is adjacent to the dorsalis pedis artery 25.

The description of the array component 10 as being “adjacent” to the dorsalis pedis artery 25 means that the sensor array component 10 is placed in as area, generally designated at ‘B’ in FIG. 1A, such that the sensor 1 array 4 can detect cardiac output artifacts resulting from the change in pressure signals measured directly from the dorsalis pedis artery 25 at one or more of the individual sensors 3 forming the plurality of sensors of the sensor array 4. As described in more detail below, placement of the sensor array 4 proximate to the dorsalis pedis artery 25 enables the selection of at least two of the individual sensors 3 of the sensor array 4 to provide cardiac function data provided by one individual sensor 3 selected from among the individual sensors 3 of the sensor array 4 and at least one additional sensor 3 to detect a motion artifact resulting motion of the foot or from the body that can be directly detected at the foot through the use of the individual sensors 3 in the sensor array 4. As is described in more detail below with respect to the exemplary articles of footwear (see e.g. FIG. 3), placement of the sensor component 10 in an article of footwear 30 can be achieved through a number of different orientations and in a number of different configurations relative to the article of footwear 30. Moreover, the performance of the sensor array 4 and the parameters for measuring pressure sensor signals as described in the pressure sensor data disclosed herein, enables the sensor to array 4 to gather differential pressure sensor signals even with a fabric barrier, such as a sock, or then barrier layer placed between the surface of the skin around the dorsalis pedis artery 25.

Referring specifically to the physiology shown in FIG. 1B, the four major tendons originate from the following muscles: tibialis anterior (TA) 21, extensor hallucis longus (EHL) 22, extensor digitorum longus (EDLS) 23, and fibularis tertius 24. The EDLS tendon 23 has four branches that connect with four toes distally and merge into a single muscle proximally. These tendons 21,22,23,24 connecting muscles 29 from the shank to the foot bones 26, play important roles in controlling the pedal motions. When a motion is intended, one or more muscles 29 are fired to contract, and these tendons 21,22,23,24 act as flexible connections that pull the bones 26 with high strength tensile forces. Such tensile forces can also lead to skin 20 movement perpendicular to the contracting tendon, generating perceptible pressure variations over the skin 20. Therefore, the resulted pressure changes from pedal muscular/tendon motions is detected by the highly sensitive pressure sensor array 4 and analyzed as described below.

Based on the ability of the sensor array component 10 to provide signal data including motion detection from the anatomical structures described above, a novel detection methods have also been discovered based on the combination of the ability to use a wearable item of clothing such as traditional footwear to capture both cardiovascular pressures adjacent dorsalis pedis artery 25 and individual skeleto-muscular responses from the dorsum region of the foot. These methods become enabled once the sensing array component 10 as described herein has been embedded into, for example, the tongue of traditional footwear and positioned in the upper dorsum region proximate to the dorsalis pedis artery 25, as shown in FIG. 1A and FIG. 1B. As shown in FIG. 1B, this sensing region generally designated as B is located laterally above the navicular bone and three cuneiform bones. Historically, the most prominence of the navicular bone and its nearby region (within 1 to 1.5 cm) has been particularly investigated as a reliable landmark to locate the arterial pulses of dorsalis pedis artery 25. Moreover, this upper dorsum region on the foot corresponds the area between the instep and waist girth defined in the footwear industry.

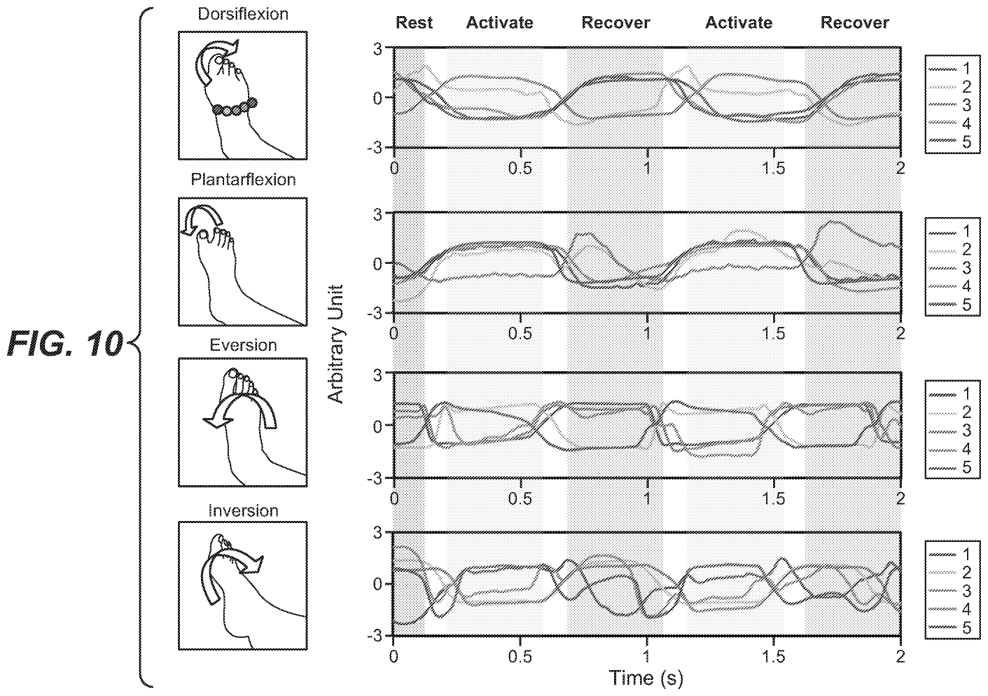

Referring specifically to FIG. 10, the sensor array 4 is shown disposed between arcs ‘C’ and ‘D’ relative to, and incorporated into, the structure of a traditional article of footwear 30. Individual structures in the article of footwear 30 are shown corresponding to the loadbearing elements of the footwear article 30. Footwear designers typically recognize that the forces exerted on an article of footwear 30 by the human foot corresponds specifically to different skeletal or physiological regions in the body. The designations reflect regions of the human foot, structural portions of footwear, and the design of an article of footwear 30 is often specifically tailored to the forces placed on the article of footwear 30 by the human foot during the process of ordinary activities such as walking, standing, and running. In FIG. 10, these areas are designated as the Waist Girth 32, the Cuneiform Bones 33, the Navicular Bone 34, and the Instep Girth 35. The location of the sensor array 4 is advantageously intermediate to these structures in the footwear article 40, rather than closer to the ankle, closer to the toe, on the side of the foot, or on the sole of the foot to advantageously take advantage of the measurements at the dorsalis pedis artery 25 and the underlying tendons and other physiological structures in the intermediate region of the foot. This location avoids the high wear conditions under the foot and bending stresses at the ankle. As this location is a relative prominence of the foot, the fabric and tightening structures of the footwear (e.g. tongue and laces) provide controlled baseline pressure to the sensor ensuring conformal contact as opposed to concave structures such as the arch which avoid such contact.

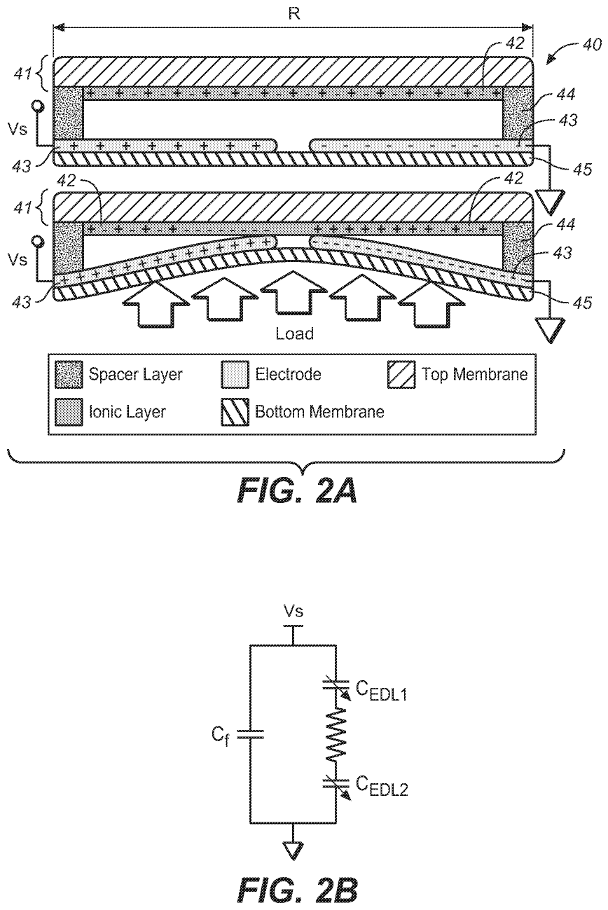

FIG. 1D is the isolated or separate sensor component 8 showing one configuration of eight individual sensors 3 formed into the sensor array 4. The sensor is comprised of a polymer substrate 1, individual flexible iontronic assemblies (FITS) as described below in FIG. 2, and electrical leads 8 disposed within the substrate 1 and communicating electrical signals from the individual sensors 3 to a connector 7, typically located at the opposite end of the sensor component 8. In one embodiment, one individual electrical lead 8 forms an electrical connection each individual sensor 3 and wherein each is oriented in a series of unidirectional parallel lines disposed in an elongated portion 5 of the substrate 1 that is integral with a portion of the substrate 1 containing the individual sensors 3 of the sensor array 4. In this configuration, the substrate 1 is highly flexible forms a waterproof, transparent, unit with minimal elasticity that contains an entire comment intact, uninterrupted, electrical connection between each individual sensor 3, via each dedicated electrical lead 8 four connection to the signal-processing circuit module (not shown—see FIG. 3 below). The portion of each individual lead 8 farthest removed from each individual sensor 3 may feature a transitional region 6 that either terminates in a connector 7 or is integrally formed with an electrical lead that is part of the article of footwear 30 and provides an operative electrical connection to the circuit module as described below. The connector 7, when present, has a fixture wherein each individual electrical lead 8 forms an electrical connection with a matching electrical lead in a receptacle (not shown) for transmission to the circuit module.

Continuing to refer to FIG. 1D, To achieve an alignment-free detection of pressure signal data proximate to the dorsalis pedis artery 25, the multi-sensor sensing array 4 is designed and oriented with each individual sensors 3 being approximately 1 cm in diameter and having a lateral spacing of 0.5 cm between the outer tangent edge of each individual sensor unit 3. In such a configuration, variations in the anatomic location of the dorsalis pedis artery 25 from person-to-person can vary within a diameter of the sensor array 4 within a range of the range from 1.5 mm to 5 mm, and with varying articles of typical footwear 30 to keep an individual sensor 3 from among the plurality of sensors in the sensor array 4 within 1 cm from the center of this artery and the cardiac signal and adjacent motion artifacts from the underlying physiology logical structures at this described in FIGS. 1A and 1B above such that useful differential pressure sensor data can readily be detected at the extra-dermal surface adjacent to the dorsalis pedis artery 25 even if the sensor array 4 is positioned in the tongue of tissue and even if the user is wearing a sock or other fabric layer intermediate to the extra-dermal surface of the artery and the sensor array 4. V. Kulkarni and B. Ramesh, “A Morphological Study of Dorsalis Pedis Artery and Its Clinical Correlation,” Int. Organ. Sci. Res., vol. 2, pp. 2278-3008, Aug. 2012.

FIG. 2A is the device architecture of FITS device 40 before and after a mechanical load is applied, which consists of a top sensing member 41 coated with an ionic layer and a bottom flexible electrode 43 having an anode and a cathode, separated by a spacer layer 44. Specifically, once a mechanical loading (P) is applied, the flexible electrode 43 will deform and create a contact with the ionic coating layer 42. This contact forms an electric double layer (EDL), where mutual attraction of both mobile electrons in a conductive solid phase and counter-ions accumulated in an adjacent ionic environment in the ionic layer 42 occurs. Therefore, upon the application of load, the FITS device assembly 40 can generate an ultrahigh interfacial capacitance between the ionic layer 42 and the flexible electrode 43. As the external pressure load rises, the contact surface area between the ionic layer 42 and the flexible electrode 43 increases as does the interfacial capacitance increases, which can be detected by the readout circuit. Notably, the unit-area value of this interfacial capacitance (C₀) is typically three to four orders of magnitude higher than that of the conventional capacitive sensors, such that the output signal of the FITS device 40 enjoys a signa-to-noise ration large enough that the sensing signal significantly overwhelming parasitic and motion artifact noises and enables separation of cardiac and motion artifact signals.

FIG. 2B exhibits an equivalent circuit model of FITS device, where C_{EDL1} and C_{EDL2} are the interfacial capacitances with an equal magnitude and Rᵢ stands for the internal resistive value of the ionic film. These interfacial capacitances are directly proportional to the contact area between the ionic and electronic surfaces, which can be calculated from the classic thin plate theory and a touch mode assumption model. Moreover, C_{f} represents the fringe capacitance between the adjacent electrode surfaces, which is much smaller than C_{EDL1} and C_{EDL2} that can be negligible in our case. As a conclusion, the total capacitance (C) of FITS device can be expressed as (the detailed derivations can be found in ESI):

$$C = {C}_{f} + \frac{{C}_{EDL 1} {C}_{EDL 2}}{{C}_{EDL 1} + {C}_{EDL 2}} = \frac{1}{2} {C}_{EDL 1} = \frac{1}{2} π {{C}_{0} ( r - \sqrt[4]{\frac{64 D \times h}{P} ( 1 + 0.488 \frac{{h}^{2}}{{t}^{2}} )} )}^{2}$$

where r and t stand for the radius and thickness of the sensing membrane, respectively; indicates the thickness of the spacer layer; D represents the flexural rigidity of the deforming substrate.

Referring specifically to the operation of the FITS device 40 in FIG. 2A, the architecture of the FITS device 40 and the performance of the iontronic sensing modality, such that the output pressure signal is based on the difference in unit-area capacitance resulting from application of load as shown in FIG. 2A to the sensor array 4 substantially enhances the overall device performance. A gel-like ionic polymeric matrix was selected as the coating material, which has been thoroughly studied in the field of solar cells and high-performance batteries, as the gel electrolyte as described in M. Watanabe, M. L. Thomas, S. Zhang, K. Ueno, T. Yasuda, and K. Dokko, “Application of Ionic Liquids to Energy Storage and Conversion Materials and Devices,” Chem. Rev., vol. 117, no. 10, pp. 7190-7239, May 2017 which is specifically incorporated herein by reference. In particular, the ionic layer 42 was prepared by mixing a polymeric matrix polyvinyl alcohol, PVA) and an ionic liquid (1-ethyl-3-methyl-imidazolium tricyanomethanide, [EMMI][TCM]), and then cured by a standard solvent evaporation procedure as described in A. L. Saroj and R. K. Singh, “Thermal, dielectric and conductivity studies on PVA/Ionic liquid [EMIM][EtSO4] based polymer electrolytes,” J. Phys. Chem. Solids, vol. 73, no. 2, pp. 162-168, February 2012, which is specifically incorporated by reference herein.

Specifically, 0.5 g PVA (341584, Sigma-Aldrich) was dissolved in 10 g distilled water. Then, this PVA solution was mixed with 0.25 g [EMMI][TCM] (IOLITEC Inc.) and stirred at 50° C. for 2 h. To form a uniform thin ionic coating layer 42, the solution was poured and spin-coated onto the surface of a polyimide film (Kapton, Dupont) at 600 rpm for 30 s, using a commercial spinner (WS-400-6NPP, Laurell). The resulted polyimide film with the ionic coating was baked at 120° C. for 2 h on a hot plate in ambient air. Subsequently, the coated polyimide film was trimmed with the designed layout using a UV laser etching (SAMURAI UV Making System, DPSS Lasers) to complete the fabrication of the top membrane of the individual sensors 3. To form the electrode 43, a 50 nm film of Au was sputter-coated onto another polyimide sheet with a sputtering machine (AUTO108, Cressington), followed by the UV laser etching to form the interdigital single-sided electrodes. Finally, a double-sided tape (467 MP, 3M) was used to create the spacer layer trimmed and applied as the spacer layer 44 to assemble both the top sensing membrane 41 and the assembly of the electrode 43 in the bottom membrane 45 layers together.

To characterize the sensor performance and optimize the sensor dimension for the application of pedal pulses sensing and muscular activities detecting, a custom test setup was constructed using a pneumatic airbag design controlled by a high-resolution manometer (840080, Sper Scientific) to apply a uniform pressure load on the FITS device 40. The capacitive change of a single sensor as assessed in real-time by an LCR meter (4284A, Agilent), while the pressure change was recorded by the manometer. Several sensor evaluation tests were conducted, including a device sensitivity test, a performance evaluation on a curved surface as well as a temperature stability test. Device sensitivity test was to investigate the relationships between the loaded pressure and the generated interfacial capacitance by adjusting different geometric parameters of the FITS device 40 sensors, i.e., radius of the sensing chamber, sensing membrane thickness, and height of the spacer layer. For the bending stability test, the sensor was attached to convex surfaces with various radii of curvatures (flat, 25 mm, 50 mm, 100 mm). The temperature stability test was operated on a hot plate and a thermocouple probe was used to monitor the temperature of the sensor, varying from 15 to 60° C.

To continuously collect and transmit the signal data from the optimized sensing array, a custom circuit was designed and built on a printed circuit board to detect the capacitive changes comprising five-unit iontronic pressure sensing array comprised of an analog front 51, five low-voltage operational amplifiers (LMV324, Texas Instruments), an 8-bit MCU with ADC component 52, a Bluetooth low energy (BLE) module 54 (CC2541, Texas Instruments), and the power management module 53 with a standard rechargeable Lithium-ion battery.

Moreover, a custom graphic user interface (GUI) was programmed in MATLAB to receive, process, and display the signals. FIG. 4 shows the block diagram of the circuitry and also shows the integration of the sensor array4 in athletic footwear 30. The invention disclosure herein includes validation of the electromechanical sensitivity of the flexible iontronic array as well as investigation of the environmental influences on the device performance together with ability of a designer to optimize and select of certain parameters for optimal performance when integrated into a wearable article. FIGS. 2C-2E illustrate the relationship between the external load and the generated interfacial capacitance from the sensors under various geometric parameters, including the radius of the sensing chamber, sensing membrane thickness, and spacer layer height. As can be seen, as the loading pressure increases, the measured capacitance will be triggered at a threshold value (once the ionic-electronic contact is initially made) and will continue rising accordingly. Specifically, four chambers with various radii, from 1.25 mm to 5 mm, have been tested, as shown in FIG. 2C in which the spacer height and membrane thickness are fixed at 50 μm and 75 μm, respectively. As expected, the sensor with largest size of sensing chamber (r=5 mm) shows the highest sensitivity (0.25 nF/mmHg, from 5 to 40 mmHg), while the sensors with smaller chamber sizes (r=2.5 mm and r=1.25 mm) may experience relatively high threshold pressure (over 100 mmHg) as well as low device sensitivity (0.01 nF/mmHg, from 100 to 200 mmHg). As shown in FIG. 2D, the membrane thickness of the sensors also plays an important role in the sensor performance. the sensor with thinner sensing membrane (of 25 μm in thickness) exhibits a sensitivity of 0.6 nF/mmHg from 5 to 40 mmHg, which is 6 times greater than that with a thicker membrane of 75 μm in thickness (0.1 nF/mmHg), given a fixed spacer height of 50 μm and chamber radius of 3.75 mm.

As shown in FIG. 2E, different spacer heights have been investigated in individual sensors 3 with a fixed chamber radius of 3.75 mm and membrane thickness of 75 μm have been investigated. The sensor with a lower spacer height (20 μm) reaches a lower threshold pressure (5 mmHg) as well as a higher device sensitivity (0.1 nF/mmHg, from 5 to 40 mmHg). These experimental data match the theoretical performance as described above. To detect cardiac function and physiological motion artifacts in a sensor array4 that is comfortably integrated into conventional footwear 30, a FITS sensor array 4 with high pressure sensitivity and low threshold pressure is required to be able to measure the differential pressures proximate to the dorsalis pedis artery 25 of under 200 mmHg. Therefore, for the applications of the sensor array 4 adjacent to the dorsalis pedis artery sensors with a radius of 5 mm for the sensing chamber in the range of 2 to 20 mm with 5 mm for the most preferred, a thickness of 25 μm for the sensing membrane in the range of 10 to 250 μm with 25 μm for the most preferred, and a thickness of 20 μm for the spacer layer in the range of 10 to 200 μm with 20 μm are preferred

The structural curvature has also been investigated on its influence over sensor performance. The measured capacitive values have only marginal changes on the surfaces with a radius of curvature varying from 25 mm to an infinite flat. This result implies that consistent device performances is obtained under different surface topologies and additional calibration steps can be optionally assessed under specified conditions and may be bypassed even in variations from use to use and from person to person and this bypass preserves power and aids in ease of use. Finally, temperature variation was studied across temperature rises from 15° C. to 60° C., at no pressure, pressure of 100 mmHg, and pressure of 200 mmHg and revealed to be less than 5%, indicating substantial immunity of the sensors to environmental temperature fluctuations.

Referring to FIG. 3, a conventional article of footwear 30 is shown having a tongue 41 a shoe shell or wall 43 and insole 44 and a sole 45. The sensor array 4 is disposed within the tongue 41 and is preferably closely conforming to the entire interior surface of the tongue 41 which may be specially sized and configured to receive the dimensions of the sensor array 4 a sensor module 42 may be located proximate to the sensor array 4 near the tongue 41 or may be placed elsewhere on the footwear 30 or may be on associated structures such as socks or laces for transmission of the data signals generated by the sensor array 4 to the graphical user interface (GUI) for perception by the user as described below. In use, pressure signals obtained from the sensor array 4 are processed via the circuitry 47 contained in the circuit case 42 and transmitted to a receiving unit (not shown) having a graphical user interface (GUI) for display to the user. The sensor array 4, can be permanently integrated into the footwear 30 either in the tongue 41 or in a separate structure that maintains the sensor component 10 proximate to the dorsalis pedis artery 25.

The sensor component 10 may also be removable, for example by a single user, and introduced into different items footwear 30 that are worn by the user. The sensor component number 10 and the circuit case 42 may be integrated or completely separable by virtue of the plug 7 (see FIG. 1D) such that a single or multiple sensor components 10 and circuit modules 42 can be used by a single user a crossed different articles of footwear 30. Furthermore, a single circuit module 42 may be used by single user with any number of sensor components 10 integrated into different articles of footwear for specific use. By this method, performance data across different articles of footwear 30 may be obtained and measured as a process for evaluating user performance or a wide variety of other cardiovascular or physiological/motion parameters with different articles of footwear 30 or under different ambient conditions.

Within the article of footwear 30 for example within the tongue 41 and enclosure may be created that has a border to engage the outer portion of the sensor array 4 or sensor component 10 to hold the individual sensors static during a sensing session. In this configuration, the outer border of the sensor component 10 would be tailored to the inner border of the compartment integrated into the article of footwear 30 so that the positioning of the sensors is reproducible. In this configuration, the flexible polymer substrate in which the sensor component 10 is disposed is positioned between two layers of an article of footwear 30 and held in close conforming engagement with the physiology of the foot adjacent to the dorsalis pedis artery 25. Although the flexibility of the sensor system does not require precise positioning of the sensor component ten during any sensing session, the orientation of the sensor component ten within the article of footwear 30 that places the largest number of individual sensors 3 of the sensor array 4 adjacent to the dorsalis pedis artery 25 improves the overall performance of the device.

Referring to FIG. 4, the five-component integrated pressure sensing and data transmission system 40 is integrated into the shoe tongue 41 of an article of conventional footwear 30 for the continuous measurement of the cardiac function and physiological artifacts adjacent to the dorsalis pedis artery 25. The block structures of FIG. 4 illustrate integration of the sensor array and circuitry 47 in a regular athletic shoe 30, where the 6 block components schematically represent the processes and structure forming the signal path from the sensing array 4, to an analog front 51 for receiving the analog pressure signal from the sensor array 4, through analog-to-digital converter and microcontroller unit 52, Bluetooth communication, preferably BLE, and antenna 54 and power management utility 53 for communication with the circuit module structure 42, for data processing and analysis.

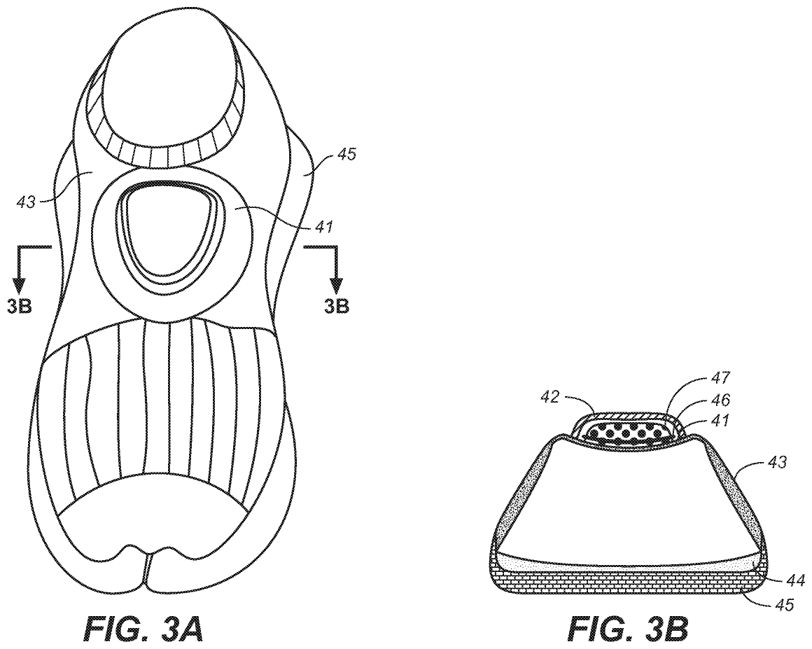

Referring to FIG. 5, an analytical flowchart 60 shows the progression of sensing steps leading to verification of separate detection of pressure signals for cardiac analysis and physiologic motion analysis. The integrated pressure sensing and data transmission system 40 pursues a progression of analytical steps to enter operation and progress through a series of steps to verify that the system 40 is in proper operation and that the sensor array 4 and sensor component 10 are properly positioning and connected to the data acquisition and processing components to collect, record, and analyze the necessary data for output to the user. Initially, the system 40 is in a standby mode 61 prior to the detection of the sensor array 4 being active and detecting data from an article of footwear 30. A first sensing step 62 determines whether or not the system 40 is in an active acquisition mode. If not, the system 40 returns to the standby mode 61. If the acquisition mode is active, the system 40 determines whether or not the sensor array 4 disposed in the footwear 30 indicates that the signal from the footwear 30 is active 63. If the system 40 indicates that the detection of signals from footwear 30 is active, analysis of data from the sensor array 4 yields a determination 64 whether or not the user is wearing the footwear 30 in a configuration that will permit collection of valid differential pressure sensor data from the sensor array 4.

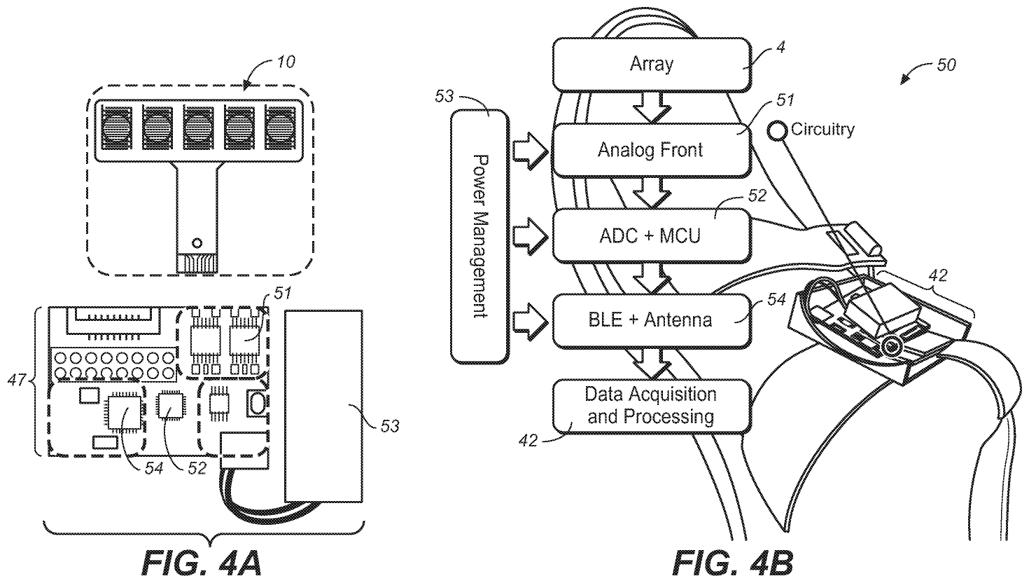

Continuing to refer to FIG. 5, verification of the user wearing the sensor array 4 in a proper orientation generates either of an error message 72 if the proper orientation is not present and the error message 72 is communicated to the GUI of FIG. 6 to alert the user. If a proper orientation of the sensor element 10 is indicated, the system 60 proceeds with a status check 66. If the status check 66 indicates that the system 60 is in a configuration for acquisition of relevant pressure sensor signaling data, the first cardia sensor and the second motion sensor are specified and the sensor array 4 is activated for the ability to assess motion artifacts for analysis 68. If motion analysis 68 is detected, the system 40 saves the results of the motion analysis 69 for further processing and resets to the detection step 63 for obtaining additional readings. If motion cannot be detected, the system 40 measures cardiac signals 74 from the first cardiac sensor and any other existing second, third, or additional and saves those results 71. Further analysis of the stored motion artifact results 69 and the stored cardiac results are further processed as described below for display to the user graphical user interface as described in FIG. 6.

As noted above and below, assignment of an individual sensor 3 from within the sensor array 4 for detection of the cardiac signal for analysis 70 as opposed to the motion artifacts for analysis 68 is arbitrary and the respective signals may be obtained from a different individual sensor 3 depending on the user, the particular article of footwear 30, even when the particular article of footwear 30 is used in a different sensing sessions from an individual user. Additionally, the step progression in the analytical flowchart of FIG. 5 between the assessment of static status 67 and the progression to motion analysis 68 a cardiac analysis 70, is not necessarily or commonly binary. The system 40 may proceed to measure cardiac pressure signals from one or a plurality of individual sensors 3 that are assigned in any particular sensing session, based on the characteristic of the data signal received, as comprising cardiac function 74 further storage and analysis 71. Similarly, the system 40 may proceed to measure motion artifact signals 68 from multiple individual sensors 3 for storage and analysis 69. In particular, once the system determines at the assessment of a static state 67 that motion analysis is occurring, repeated detections of motion analysis 68 based on differential pressure signals from any individual sensor 3 or the combination of sensors in the sensor array 4 leads to the complex analysis of cardiac and motion functions as described below.

FIGS. 6A-6C illustrate a series of graphical use interfaces showing several cardiac and physiological parameters measured by the sensor array and displayed to the user. In the panel of FIG. 6A, an example of a visual representation of pressure sensor signals from the sensor component 10 as analyzed and displayed to the user results from a user at rest and in a sitting position. A numerical heart rate value is displayed together with a respiratory rate “BR,” a blood pressure value “BP” an indication of an activity, or activity level of the user “SIT.” The lower panel shows separate readings for continuous graphics for pulse and respiration. The middle panel of FIG. 6B is representative of a more active state. The heart rate, respiration rate BR, blood pressure value “BP” and status indicator are measured or changed appropriately in response to pressure signals from the sensor array 4. The lower panel of FIG. 6B also reflects the change in activity of the user. The GUI may display any or all of cardiac data, respiration rate, foot gestures, motion status, general or specific activity levels and comparisons of all of the parameters discussed herein with prior user sessions or with stored guidelines. The graphical user interface is also capable of displaying a specific calculations that are pre-loaded in the signal analysis portion of the data processing function to calculate pre-existing parameters based on data from the sensor component 10 and, potentially including other conventional sensors that may be integrated into the data processing functionality. Such additional sensors include conventional pressure, temperature, optical sensors, accelerometers, and the like.

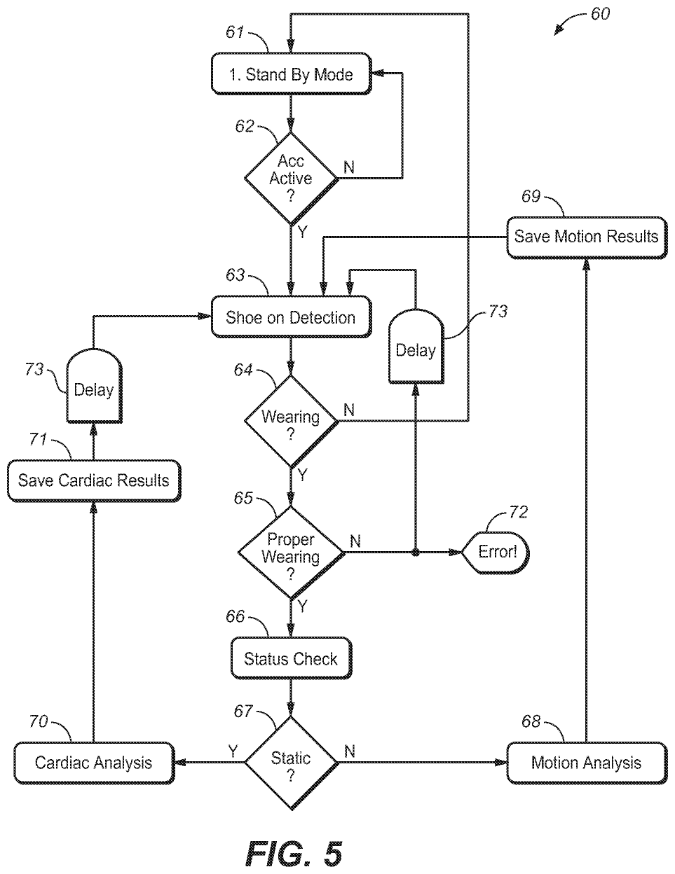

Measurement results can also provide the information about respiratory patterns together with cardiac function, as shown in FIG. 7. It has been known that respiratory activities would modulate the arterial pulse waveforms, from which the respiratory rates can be potentially extracted from the arterial pulse waveforms due to the vasomotor response. From the signals acquired by the device, the contour of the arterial pulse waveform implies a respiratory rate of 12 per minute, indicating the potential of the sensor array of the invention and integrated, multiplex system to track more than one vital signal by using one single device.

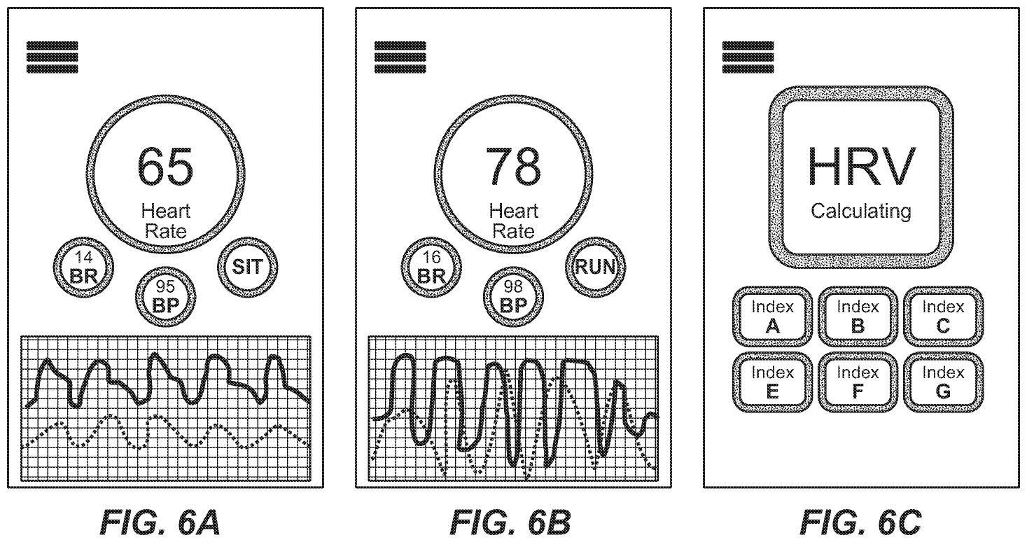

Referring to FIG. 8, a typical high-resolution pulse waveform recorded from the system 40 adjacent to the dorsalis pedis artery 25 from which the pulse waveform patterns can be extracted from the waveform envelope. Points i-v refer to i) diastolic uprising, ii) systolic peak point, iii) systolic decline, iv) valley and v) peak of peripheral dicrotic notch, respectively. From the recorded pulse waveform, the characteristic features during each cardiac cycle can be extracted and related to the corresponding cardiovascular events. These feature points can be used to calculate more valuable or indicative cardiovascular parameters, such as upstroke time (Tup) and augmentation index (AIX) as follows:

$${T}_{up} = {t}_{ii} - {t}_{i} \qquad ( 2 )$$

$${AI}_{X} = \frac{{P}_{iii} {P}_{i}}{{P}_{ii} - {P}_{i}} \times 100 \% \qquad ( 3 )$$

where tₓ and Pₓ indicate the corresponding time and pressure value of each individual feature point i, respectively, respectively. The high-resolution pulse waveform of FIG. 8 enables real-time monitoring of cardiovascular activities on a continuous basis with potential preventive features. For example, typical patients with peripheral artery disease (PAD) have elongated Tup in pedal pulse waveforms, while the peripheral AIX has been investigated to correlate with cardiac risk factors and coronary artery diseases (CAD).

Moreover, a custom HR-detection algorithm has been implemented by using a peak-detection method. Specifically, following the collection of the pedal pulse signals from the device, any signal or collection of signals are fed through the MATLAB algorithm consisting of a band pass filter from 0.1 Hz to 5 Hz and a peak detection module to detect the systolic peaks of the pulse waveform in the time domain, from which the real-time HR can be computed continuously every second. Meanwhile, a standard ECG electrode pair has been placed on the right arm, left arm and left leg of the same testing subject and the cardiac bioelectrical signal has been simultaneously acquired, which is considered as a golden standard for HR detection in clinic practices, to calculate the accuracy of the HR from the real-time pulse waveform from the device of the invention.

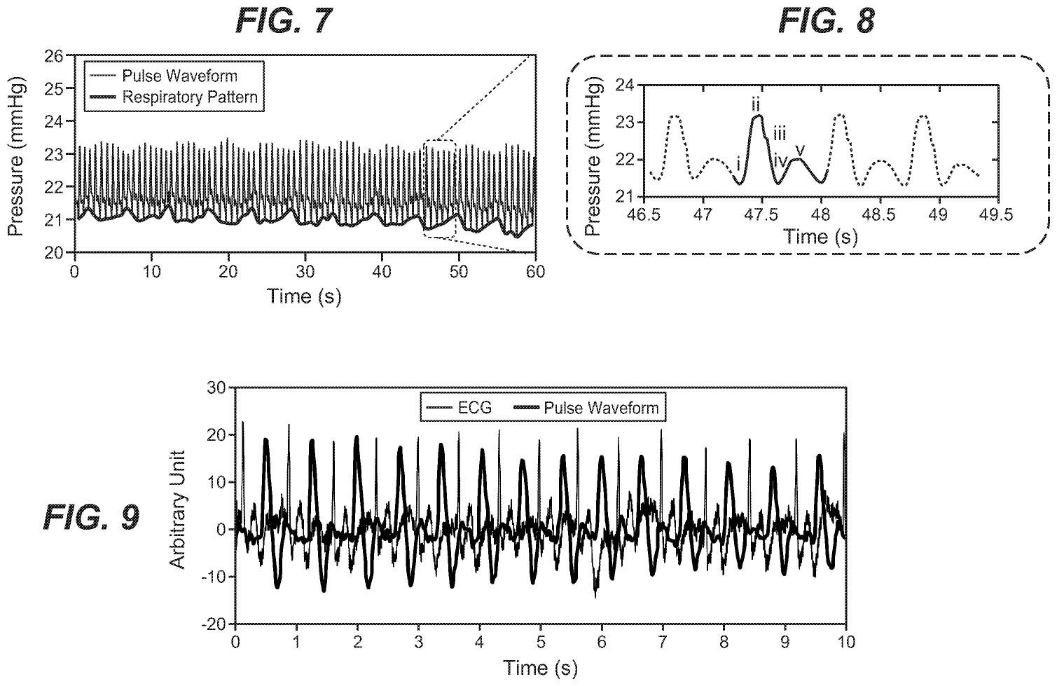

FIG. 9 plots the simultaneously recorded the pulse waveform (bold) and the ECG waveform (fine) normalized to the same scale. From the recordings, the two signals, one representing the bioelectric events associated with the cardiovascular pace-making and the other corresponding to mechanical movements of the blood circulation, are highly correlated with each other, with a correlation coefficient of 0.97.

Moreover, a Bland-Altman analysis reveals a low mean error of 0.81 bpm between ECG and the output of the invention with a 95% confidence interval from −0.74 BPM to 2.4 BPM. These result indicates that when analyzing measurements from these two devices (ECG vs sensor array) in a static condition, high accuracy can be acquired. This level of accuracy is within the published standard for heart rate measurement, the standard for cardiac monitors, heart rate meters, and alarms (ANSI/AAMI EC13:2002). These measurement results show the potential of the HR measurement from the device to be within a medical-grade precision as confirmed by ECG and pedal pulse waveforms normalized at the same scale and the correlation (r=0.97) between the heart rate calculated from the sensor array 4 of the invention and the heart rate from ECG and based on a Bland-Altman plot of heart rate HR comparison between a value calculated from the sensor array 4 and a value from ECG.

In addition to the vital signal extractions and arterial pulse waveform measurements, wearable tracking of skeletomuscular activities can be implemented using the flexible sensing array, in which individual tendon movements have been investigated. According to human anatomy adjacent to the dorsalis pedis artery 25 as shown in FIG. 1A, pressure signals from the four major tendons (TA, EHL, EDLS, and FT) are covered and measured by the individual sensors 3 adjacent in the dorsum region. The position of the dorsalis pedis artery 25 is located during the process of identifying cardiac signals in the process as described in FIG. 5, and as a result, the location of signals form the individual sensors 3 enables a natural spatial reference to determine the relevant tendon locations within the relative range of separation of these anatomic structures for a typical human subject. As shown in FIG. 10, channel #2 is recognized as the location of the dorsalis pedis artery 25 from the pressure signals generated from the sensor array 4 and compared to a stored cardiac profile. As described in more detail below, following the initial detection of a cardiac signal 70, the spatial orientation of at least the 4 major tendons are referenced for potential signal processing 69 and storage for further analysis, in this process, the motion artifacts measured in four sensing channels (1,3,4,5) in FIG. 10 can be directly related to the activities of the individual tendons, for example referring again to in FIG. 1A, as Channels #1 for TA, #3 for EHL, #4 for EDLS, and #5 for FT.

Referring to FIG. 10, big toe dorsiflexion, the contraction of EHL tendon causes the big toe to bend up. This contraction also lifts the position of EHL towards the shoe tongue and thus increases pressure received by the specific sensing channel. As can been seen, Channel #3 receives pressure variations from EHL shows a signal increase during the active phase and a signal decrease during the recovery phase, compared with other four channels. Like big toe dorsiflexion, in big toe plantarflexion, signal from channel 3 can also be easily differentiated from others, due to the elongation of EHL. The elongation of EHL decreases the pressure applied the channel 3 during the active phase. In foot eversion, the elongation of FT enables the signal of channel 5 in (FT) to rise due to the pressure increase in the lateral side of the dorsum region. While, in foot inversion, both of the signals from channel 1 in (EDLS) and channel 5 (FT) follow the similar trend, as both of EDLS and FT are involved in. Based on pressure values that each channel received during the active phase, a rule based fuzzy logic table is summarized in Table I. Using this table, each of the four foot gestures can be distinguished. Therefore, the sensor array can be utilized to detect individual tendon responses as well as pedal gestures. It also shows great potential to be applied in everyday activity tracking in a natural and unnoticeable fashion.

TABLE I
RULE BASE OF FUZZY LOGIC TO DISTINGUISH GESTURES

| Foot gesture | 1 | 2 | 3 | 4 | 5 |
|---|---|---|---|---|---|
| Dorsiflexion | Low | High | High | Low | Low |
| Plantarflexion | High | High | Low | High | High |
| Eversion | Low | High | Low | Low | High |
| Inversion | High | Low | Low | Low | High |

A highly sensitive and flexible pressure sensing array enabled by the novel iontronic sensing principle has been fabricated, to simultaneously acquire body vital signals as well as track pedal skeletomuscular activities, which has been seamlessly integrated in a footwear format such as a shoe. The device demonstrates that a foot wearable device can capture high-resolution peripheral arterial pulse waveforms, from which both heart rates and respiratory patterns can be extracted within a medical-standard precision. Moreover, the high-spatial resolution of the sensing array allows alignment-free capture of pulse signals as well as provides a spatial reference to the pedal structures. It further enables tracking of individual pedal tendon movements, from which the majority of foot gestures can be assessed in real-time. The device also enables a valuable personal mobile platform to acquire and analyze the human health and activity information in a comfortable and unnoticeable fashion.

Individual datasets are assembled from the individual sensors 3 in the sensor array 4 and stored for processing or analyzed in real time against stored sensor data. These datasets include a first signal dataset from a first pressure sensor in the array. As noted above, the designation of an individual sensor as first, second, or third etc. is arbitrary and only refers to individual sensors 3 of the sensor array 4 that are analyzed in a sensing session and assigned a status of first, second, third etc. in the pressure signal processing steps, the individual datasets may include each of discrete cardiac datasets, static datasets, inertial datasets and combinations of each. Each dataset from an individual sensor may be assigned as yielding any of the cardiac, static, or inertial datasets or combinations thereof. Furthermore, the datasets may be correlated to spatial orientation based on the known spacing, orientation, and distances between the individual sensors 3 in the sensor array 4. Based on this orientation, individual pressure signals may be comprised of a component that indicates the spatial relationship to any individual sensor 3 in the sensor array 4 or any combination thereof. The data processing system may contain stored data values for a sample cardiac profile that includes the artifacts described above for comparison with the sensed pressure signal from any component of the sensor array 4 including combinations of individual sensors 3 in the array 4. A cardiac profile may also be created from prior sessions wherein a user creates a cardiac profile by wearing the item of footwear 30 together with the sensor array 4 during a static or sensing shut session as described in FIG. 5.

Because of the ability to correlate pressure sensor data with physiological structures based on the known orientation between the dorsalis pedis artery 25 and the major muscles, bones, and tendons of the foot, as shown in FIG. 1A, individual components of a dataset may include identification of specific tendons in the foot that contribute to motion data. In addition to pressure sensor data that comes from the sensor array 4 of the invention, the data processing functionality may incorporate data from non-pressure sensors, including temperature, accelerometers, gyroscopes, magnetometers, light detectors and individual and pluralities and combinations thereof.

The methods of the invention enable the detection and measurement of a number of cardiac and physiological parameters resulting from pressure sensor signals generated by the array when the sensor component generated by the array is placed proximate to the dorsalis pedis artery. Pressure sensor data is obtained from at least two of the individual sensors in the array based on changes in pressure sensed proximate to the dorsalis pedis artery. In the data processing methodology, a first pressure sensor is identified from amongst the individual sensors in the array and one sensor is identified as the source of a primary cardiac data signal. A second signal is identified as providing additional sensor data that may be supplemental cardiac data or may be a result of sensing pressure differences from physiological changes proximate to the dorsalis pedis artery. These physiological changes result from the motion of muscles, tendons, bone, interstitial tissue, cartilage and other structures that can be translated based on changes in the pressure sensor signals.

The method of the invention includes placing the sensor component into an article of footwear and making a connection to a circuit module that enables detection storage and processing of the sensor data as described above. The data processing includes using the known spatial orientation of the individual sensors in the array to identify particular physiological structures as described in connection with the description of FIGS. 1A and 1B. The data processing includes correlating individual sensor data, such as from the primary cardiac signal and a second sensor that includes physiological data to translate the pressure signals into a readout that can be viewed by the user in any of a digital, analog or complex format. The data processing includes comparing pressure sensor data from sensors assigned to measure physiological data with sensors assigned to measure a cardiac data profile and comprises the individual steps of assigning individual sensors in the array to either of cardiac or physiological data.

The methodology of the invention also includes identifying a particular individual sensor as the primary cardiac signal as described above through comparison with a stored cardiac data profile including a stored cardiac data profile from prior uses of the sensor array by the same user. The methodology of the invention also includes determining a specific distance between a pressure sensor identified as the primary cardiac sensor and at least one additional sensor in the array based on a known specific distance between two sensors based on the orientation and spatial relationship of individual sensors within the overall sensor array as fixed in the sensor component. The orientation and spatial relationship of sensors in the array can also be correlated with the physiological profiles of the human foot and individual physiological structures can be assigned as being measured by the second or supplemental pressure signals detected by the second or supplemental sensors in the array following identification of the primary cardiac signal.

As described in connection with FIG. 10 above, the methods of the invention also include using the sensed pressure signal data to identify a specific foot gesture based on pressure sensor data received from the array and processed in the circuit module. The signal components include all of isolated pressure sensor data, primary and secondary cardiac data, individual and a plurality of motion signals corresponding to movement of physiological structures, a static signal indicating the lack of motion, taken in isolation or coupled with the cardiac output signal to either verify proper operation of the integrated sensor component or to determine a status of activity of the user. The sensor data may also comprise and inertial signal and combinations of any of the foregoing processed to generate an output for perception by the user. The output may be in the form of a visual display such as in a graphic user interface, and audio signal, or a sensory signal such as vibration that indicates status or performance of the integrated sensor component to the user.

Data processing techniques that are unique to the sensor array of the invention include using the first second, or any sensor as a noise detection function to separate out extraneous pressure sensor input from the primary cardiac and additional sensor inputs. The calculation of any value resulting from the sensory input can be obtained by addition, subtraction, summation, multiplication or other manipulation of the data to yield an output perceived by the user. The output includes a measurement of all of pulse, heart rate variability, blood pressure, foot gesture, respiration and respiration patterns, cardiovascular system parameters such as blood pressure, pulse, pulse flow, including arterial system parameters such as arterial integrity, arterial patency, arterial flexibility, vascular system parameters, detection of heart valve operation, including valve patency and related parameters, and combinations thereof. Also, cardiac abnormalities such as arrhythmia and tachycardia can be detected. Any of the foregoing parameters can also be detected and upper and lower limits established wherein touching either the upper or lower limits generates a separate signal to the user.

In addition to sensing foot gestures, data output from the sensor component can be translated into interpretations of the activity level of the user indicating rest or recline, activities such as sitting, walking, and running and can be used to correlate any activity mode to any of the measured cardiac or physiological mode such that characteristic motion, cardiac, and respiratory patterns can be assigned to different activities by the user such as sitting, walking, and running.

Calibration of the overall system as well as the individual sensor component can be achieved by repeated use by a single user wherein the data from a single sensing session is compared to a stored data profile such that future sensing sessions for an individual user can be correlated according to detection of input from the primary cardiac sensor the secondary physiological sensors and combinations of the above. Calibration sessions can also be performed between individual sessions by the user and correlated to changes in any of the cardiac or physiological data as measured.

Although the disclosed examples have been fully described with reference to the accompanying drawings, it is to be noted that various changes and modifications will become apparent to those skilled in the art. Such changes and modifications are to be understood as being included within the scope of the disclosed examples as defined by the following claims.

We claim:

1. A sensor apparatus configured for measuring physiological data proximate to the dorsalis pedis artery comprising:
a flexible sensor array comprising a plurality of electrically interconnected pressure sensors sized to engage an inner portion of an article of footwear having means to maintain the sensor array adjacent to the dorsalis pedis artery;
a circuit having a processor and data communication means for relaying sensor data from the flexible sensor array to user access means.

2. The sensor array of claim 1 wherein the at least two signal datasets generated by the sensor array and processed by the circuit module comprise:
a first signal data set from a first pressure sensor of the array containing first cardiac data, static data and inertial data; and
a second signal dataset from a second separate pressure sensor of the array containing a pressure sensor data selected from the group consisting of a second cardiac data, motion data from a physiologic structure, a static data and inertial data and combinations thereof.

3. The sensor array of claim 1 further comprising a third sensor and a third dataset comprising reference data, static data, and inertial data.

4. The sensor array of claim 1, further comprising a layered structure adjacent the flexible sensor array that engages the article of footwear to maintain the sensor array in conforming engagement with the dorsalis pedis artery.

5. The sensor array of claim 1 wherein the second signal dataset identifies a specific tendon in the human foot that contributes to motion data.

6. The sensor array of claim 1, wherein the plurality of sensors are maintained in a flexible polymer substrate sized to be positioned between two layers an article of footwear.

7. The sensor array of claim 1, wherein each sensor sensitivity in the range of 0.01 nF/mmHg to 1 nF/mmHg, detection range of 1 to 200 mmHg, response time range of 0.1 to 10 milliseconds, resolution range of 0.01 mmHg to 1 mmHg, and covers at least 10 square millimeters area and no large than 3 square centimeters.

8. The sensor array of claim 1, wherein the plurality of sensors is contained within an area no larger than 3 square centimeters and wherein the individual sensors are spaced apart from each other by at least 200 micrometers.

9. The sensor array of claim 1, wherein at least two sensors of the plurality of sensors have of 0.01 nF/mmHg to 1 nF/mmHg, detection range of 1 to 200 mmHg, response time range of 0.1 to 10 milliseconds, resolution range of 0.01 mmHg to 1 mmHg, and covered at least 10 square millimeters area and no large than 3 square centimeters.

10. The sensor array of claim 1, wherein the individual sensors of the flexible sensor array are no thicker than 2 millimeters having both a substantially planar configuration prior to engaging the dorsalis pedis artery and a confirmed configuration after engaging the dorsalis pedis artery.

11. The sensor array of claim 1, further comprising a second data generating device comprised of a non-pressure sensor selected from the group consisting of temperature sensor, accelerometer, gyroscope, magnetometer, light detector, and combinations thereof.

12. The sensor array of claim 1 further comprising a graphical user interface comprising visual display means for indicating operability of the plurality of sensors in the sensor array.

13. The sensor array of claim 12 wherein the GUI displays cardiac data, breath rate, foot gesture, motion status, and activity levels.

14. The sensor array of claim 1, further comprising an article of footwear having means to secure the sensor adjacent to the dorsalis pedis artery.

15. A method to measure a physiological parameter comprising:
placing a flexible pressure sensor array proximate to the dorsalis pedis artery;
obtaining pressor sensor data from at least two pressure sensors in the flexible array from changes in pressure proximate to the dorsalis pedis artery;
identifying at least a first pressure sensor in the array as generating a primary cardiac data signal identifying a second signal from a second pressure sensor in the array having a known spatial relationship with the first pressure sensor;
wherein a data signal from the separate second pressure sensor is generating data a selected from the group consisting of a second cardiac data signal, movement of a tissue structure, pressure exerted by a shoe and combinations thereof.

16. The method of claim 15 further comprising the step of correlating the primary cardiac data signal and the separate second pressure data signal to generate a measure of a physiological function.

17. The method of claim 15 further comprising based on comparing pressure sensor data signal from any of the at least two pressure sensors in the array with a cardiac data profile

16. The method of claim 15 further comprising using the known spatial relationship between the first sensor and the separate second sensor in the array based on a physiological profile of the human foot.

17. The method of claim 15 further comprising determining a source physiological structure generating the motion signal based on the spatial relationship between the first sensor and the separate second sensor.

18. The method of claim 15 further comprising identifying a foot gesture.

19. The method of claim 15 further comprising identifying a signal component from the first sensor selected from the group consisting of cardiac signal, static signal, and inertial signal and combinations thereof.

20. The method of claim 19 further comprising identifying a signal component from the separate sensor selected from the group consisting of a secondary cardiac data, a motion signal, a shoe pressure data, a static signal, and inertial signal, and combinations thereof.

21. The method of claim 20 comprising subtracting the signal component from the separate second sensor from the signal component of the first sensor

22. The method of claim 21 comprising subtracting the signal from the first sensor from the separate second sensor.

23. The method of claim 15 further comprising calculating a physiological parameter selected from the group consisting of pulse, heart rate variability, blood pressure, gesture, respiration pattern, arterial system parameters, vascular system parameters, and movement patterns.

24. The method of claim 15 wherein the tissue structure is selected from the group consisting of tendon, muscle, bone, cartilage, or ligament and combinations thereof.

25. The method of claim 15 wherein the physiological function is a cardiac function.

26. The method of claim 15, wherein the cardiac function is selected from the group consisting of blood pressure, pulse, pulse flow, arterial integrity, arterial patency, arterial flexibility, cardiac valve function, cardiac valve patency, cardiac arrhythmia, and tachycardia and combinations thereof.

27. The method of claim 25, wherein the blood pressure and pulse measurements of cardiac function include upper and lower boundaries.

28. The method of claim 15 wherein the primary cardiac data signal and the separate second pressure data signal generate an indicator of proper or improper function of the sensor array.
